(19) United States
(12) Patent Application Publication
(10) Pub. No.: US 2010/0113916 A1
(43) Pub. Date: May 6, 2010

(54) Systems and Methods for Endotracheal Tube Positioning

(75) Inventor: Avinash B. Kumar, Coralville, IA (US)

Correspondence Address: MCANDREWS HELD & MALLOY, LTD, 500 WEST MADISON STREET, SUITE 3400, CHICAGO, IL, 60661, US

(73) Assignee: Avinash B. Kumar, Coralville, IA (US)

(21) Appl. No.: 12/261,268

(22) Filed: Oct. 30, 2008

(51) Int. Cl. A61B 6/12; A61M 16/04

(52) U.S. Cl. 600/424; 128/207.14

(57) ABSTRACT

Certain embodiments of the presently described technology provide methods and systems for positioning of an endotracheal tube. Certain embodiments provide an endotracheal tube system including an endotracheal tube and a removable positioning member. The endotracheal tube is sized and adapted for providing airway maintenance during an endotracheal procedure. The removable positioning member is sized and adapted to be insertable into and removable from the endotracheal tube. The removable positioning member includes a positioning element located proximal to the distal end of the removable positioning member. The positioning element provides an indication of position when at least a portion of the endotracheal tube is inside of a patient.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

An endotracheal tube (“ETT”) is a medical device used for airway management and ventilation. Endotracheal tubes are used to maintain a definitive and patent airway in patients undergoing, for example, general anesthesia procedures and patients requiring mechanical ventilation. In use, an endotracheal tube is generally positioned in the trachea of a patient to provide an airway to the lungs. The insertion of an endotracheal tube into a patient is referred to as intubation, and the removal of an endotracheal tube from a patient is referred to as extubation.

Some endotracheal tubes include an inflatable cuff positioned near the distal end of the tube. After the tube is positioned, the inflatable cuff is inflated via a secondary lumen, through which air is provided to inflate the cuff, extending the cuff radially outward until it contacts the trachea, thereby acting to block the area between the outside of the endotracheal tube and the trachea, preventing the passage of air through that area. With the tube positioned and the cuff inflated, the ventilation of the patient can be supplied and controlled entirely through the endotracheal tube. Some endotracheal tubes that include inflatable cuffs also include an additional lumen that provides a conduit for suctioning airway secretions that may accumulate about the inflated cuff.

Generally, the preferred position for an endotracheal tube is such that its tip is located approximately 4-6 cm above the branch point of the trachea, also known as the carina. Studies have shown that positioning the endotracheal tube cuff in the suprasternal notch offers a high degree of certainty that the tube is not inserted too deeply. Incorrect positioning of an endotracheal tube can have serious consequences—inserting an endotracheal tube too deeply or too shallowly can have life threatening consequences, especially if placed incorrectly in a critically ill patient. For example, when an endotracheal tube is inserted too deeply (for example, into one of the mainstem divisions of the trachea), unequal ventilation can occur. Such unequal ventilation can decrease the oxygen carrying capacity of the blood by pathophysiologic processes, including ventilation perfusion (V/Q), mismatch, and shunt.

Some endotracheal tubes are of a double lumen design, which may be used for lung isolation procedures in thoracic surgery. Double lumen tubes contain two lumens to allow preferential ventilation of different lung segments and/or to enable the collapse of a lung for a surgical procedure. One of the double lumens is typically longer than the other, and is designed to extend into one of the lungs (most commonly the left lung) below the carina, while the first lumen terminates in the trachea. The double lumen tube contains a tracheal cuff (that expands to contact the trachea) and; on the longer of the two lumens, a bronchial cuff (that expands to contact a main bronchus). Problems can arise, for example, when both lumens are positioned beyond the carina and into the same main bronchus, or if both lumens are positioned in the trachea above the carina. If the end of the double lumen is malpositioned, it can result in inadequate deflation of the lung and/or sever hypoxemia due to ventilation mismatches. Further, if the bronchial cuff is not properly positioned, it can result in herniation of the bronchial cuff.

Other potential problems with positioning of endotracheal tubes exist. For example, a patient's body position while under anesthesia (while intubated) may be changed, such as to a lateral position. This changing of a patient's body position may cause the endotracheal tube to move from its original, appropriate position. This malposition may go undetected, creating a problem that healthcare workers are not aware of during a medical procedure.

Current methods of positioning endotracheal tubes include, for example, direct laryngoscopy, which, however, cannot always determine the position of the tip of the endotracheal tube at the time of intubation. Other current methods include listening to bilateral and equal breath sounds, as well as performing a ballottement test for the endotracheal tube cuff. When these and other conventional techniques are used, a chest X-ray may be required after intubation to confirm the position of the endotracheal tube and to exclude mainstem intubations, increasing cost and the number of procedures required for the patient. Such X-ray confirmations produce a time delay caused by the time required to take the X-ray and for a radiologist to read the X-ray; add cost for the X-ray equipment, technicians, and radiologists; and require the patient to be moved, thereby increasing the risk of dislodging the endotracheal tube, as well as any other medical device that also may be in use with the patient. Moreover, a patient's position may be moved after the X-ray, resulting in an undetected malposition of the endotracheal tube, and/or requiring an additional X-ray confirmation, further increasing delays, costs, and potential patient harm.

Some current methods of positioning endotracheal tubes include modifications to the endotracheal tubes, such as by incorporating radiofrequency, electrical, or optical components into the body of the endotracheal tube. One disadvantage of these systems is that they are built into the body of the endotracheal tube. Thus, for example, they are not easily replaceable or repairable. A malfunction of the incorporated component could have a serious adverse effect on the functioning of the endotracheal tube. Additionally, a patient may bite down on the endotracheal tube while it is in place, causing damage to the components and risking other serious adverse effects and/or costs.

Endotracheal tubes may also be used during tracheostomy procedures. A routine tracheostomy normally involves the retraction of the endotracheal tube out from the trachea in a gradual manner, to allow a surgeon to puncture and enter the trachea. This retraction is often performed with the aid of a fiberoptic bronchoscope. Improper positioning of the endotracheal tube and/or fiberoptic bronchoscope during such a procedure can result in a number of problems, including accidental extubation. Another possible adverse effect is inadequate endotracheal tube withdrawal and damage to the tube, including, for example, puncture of the inflatable cuff. Further, the fiberoptic bronchoscope may also be damaged during such a procedure, which can require costly and time-consuming repairs to the fiberoptic bronchoscope.

In light of the disadvantages and issues within the medical field noted above regarding endotracheal tubes, there is a need for an improved system and method for positioning such tubes, which previously has been unforeseen.

### BRIEF SUMMARY OF THE INVENTION

Certain embodiments of the presently described technology provide methods and systems for the positioning of an endotracheal tube.

Certain embodiments provide an endotracheal tube system including an endotracheal tube and a removable positioning member. The endotracheal tube is sized and adapted for providing airway maintenance during an endotracheal procedure. The removable positioning member is sized and adapted to be insertable into and removable from the endotracheal tube. The removable positioning member includes a positioning element located proximal to the distal end of the removable positioning member. The positioning element provides an indication of position. The indication of position may be observable outside of a patient's body.

In certain embodiments, the positioning element emits light visible through a patient's body. Also, in certain embodiments, the endotracheal tube system includes a light source attachable to and detachable from the removable positioning member, with the light source providing the light emitted from the positioning element.

Further, in certain embodiments, the removable positioning member includes a cable comprising a fiber optic channel and an insulation sleeve surrounding a portion of the fiber optic channel. The cable has an insulated portion through which light does not pass and an uninsulated portion through which light does pass, and the uninsulated portion is located proximal to the distal end of the positioning member.

In certain embodiments, the endotracheal tube includes a main passageway and an auxiliary passageway. The main passageway is sized and adapted for providing airway maintenance during an endotracheal procedure, and the auxiliary passageway has a smaller cross sectional area than the main passageway. The removable positioning member includes a cable sized and adapted to be insertable into and removable from the auxiliary passageway.

Further, in certain embodiments, the endotracheal tube may include an inflatable cuff located proximal to a distal end of the endotracheal tube, and the removable positioning member may be sized and adapted so that the distal end of the removable positioning member is positioned proximal to the inflatable cuff when the removable positioning member is fully inserted into the endotracheal tube. Moreover, the removable positioning member may include a cable sized and adapted to be insertable into and removable from the auxiliary passageway, with the positioning element adapted to emit light visible through a patient's body, and the auxiliary passageway located to provide an opening approaching the inflatable cuff such that light emitted from the positioning element passes through the inflatable cuff when the removable positioning member is fully inserted into the endotracheal tube. Further, the auxiliary passageway may be sized and adapted to provide for suction removal of secretions.

In certain embodiments, the endotracheal tube may include a first air passageway, a second air passageway, and an auxiliary passageway. The first and second air passageways are sized and adapted for providing airway maintenance during an endotracheal procedure. The auxiliary passageway has a smaller cross sectional area than the main passageway. The removable positioning member comprises a cable sized and adapted to be insertable into and removable from the auxiliary passageway.

Certain embodiments of the present technology provide an endotracheal tube positioner including a positioning member sized and adapted to be insertable into and removable from an endotracheal tube. The positioning member has a distal end, and the positioning member includes a light emitting positioning element proximal to the distal end. The positioning element may emit light visible through the body of a patient when the positioning member is inserted into the endotracheal tube that has been placed into the body of a patient. Further, the positioning member may be sized and adapted to be insertable into and removable from a suction lumen of the endotracheal tube. Moreover, the endotracheal tube positioner may include a light source attachable to and removable from the positioning member. The positioning member may be sized and adapted to extend proximal to an inflatable cuff when inserted into the endotracheal tube, such that the light emitted from the positioning element when the positioning member is inserted into the endotracheal tube passes through and/or reflects off of the inflatable cuff.

Certain embodiments of the presently described technology provide a method for identifying the position of an endotracheal tube in a patient, including advancing a removable positioning member into a known position in the endotracheal tube; providing a light emitting from a distal portion of the removable positioning member; observing the light emitted from the distal portion of the removable positioning member; identifying the position of the endotracheal tube; and removing the removable positioning member from the endotracheal tube. The light emitted may be observable through the body of the patient, and the position of the endotracheal tube may be identified based on the location of the light emitted through the body of the patient. In certain embodiments, the known position is proximal to the location of an inflatable cuff located proximally to the distal end of the endotracheal tube. Further, the endotracheal tube may be positioned such that light emitting from the known position proximal to the location of the inflatable cuff passes through the body of the patient proximal to the patient's suprasternal notch.

In certain embodiments, the method includes adjusting the position of the endotracheal tube, for example, to accommodate a tracheostomy after identifying the position of the tracheal tube; and puncturing the trachea at a distance away from the light emitted through the body of the patient.

In certain embodiments, the removable positioning member is advanced into an auxiliary passageway of the endotracheal tube. Also, in certain embodiments, the removable positioning member is advanced into a suction lumen of the endotracheal tube.

### BRIEF DESCRIPTION OF SEVERAL VIEWS OF THE DRAWINGS

FIG. 1 illustrates an endotracheal tube system according to at least one embodiment of the present technology, with a positioning member of a positioning system shown removed from an endotracheal tube.

FIG. 2 illustrates the endotracheal tube system of FIG. 1, with the positioning member advanced into the endotracheal tube.

FIG. 3 illustrates a cross-section taken along line 3-3 of FIG. 1 (additionally illustrating when the positioning member is advanced into the endotracheal tube).

FIG. 4 illustrates an elevation view of a distal portion of the endotracheal tube of FIG. 1.

FIG. 5 illustrates a cross-section taken along line 5-5 of FIG. 1.

FIG. 6 illustrates a double lumen endotracheal tube system according to at least one embodiment of the present technology, with a positioning member of a positioning system shown advanced into the endotracheal tube.

FIG. 7 illustrates a cross-section taken along line 7-7 of FIG. 6.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Certain aspects of the presently described and claimed technology provide one or more systems and methods for positioning an endotracheal tube that are easily and conveniently visually verifiable in real time; and/or allow dynamic positioning of an endotracheal tube; and/or provide for non-radiologic endotracheal tube positioning.

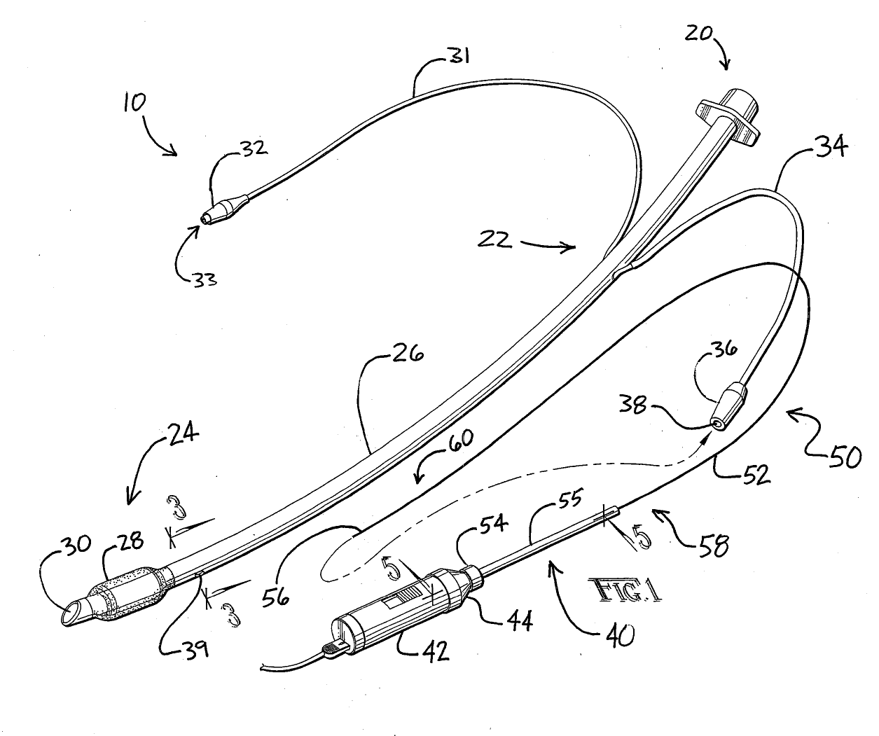

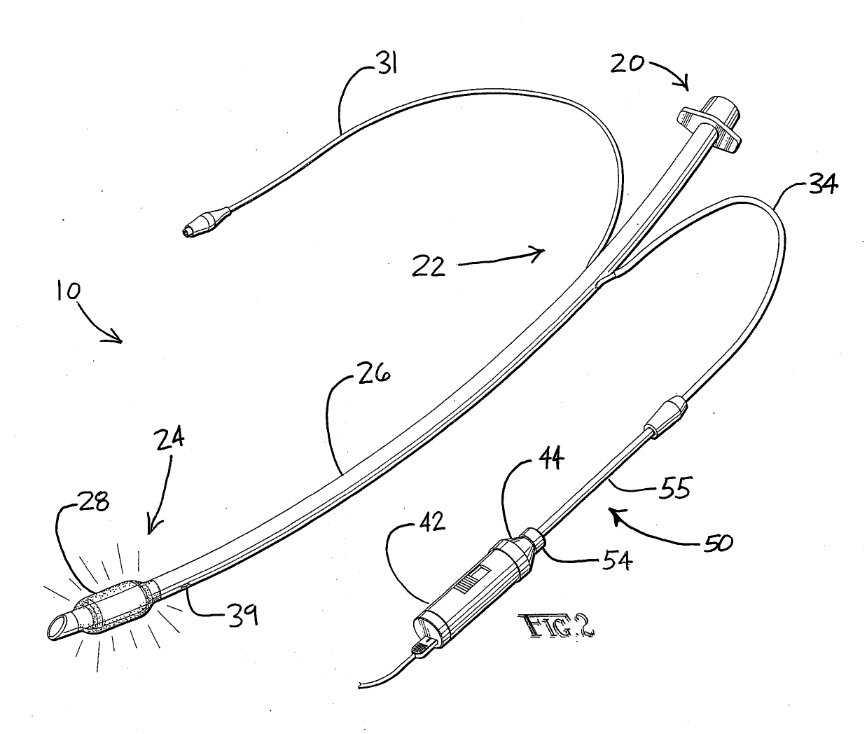

FIG. 1 shows an endotracheal tube system 10 according to at least one embodiment of the present technology. The endotracheal tube system 10 includes an endotracheal tube 20 and a positioning system 40. The positioning system 40 includes a light source 42, a light funnel 44, and a removable positioning member such as an endotracheal tube positioner 50 as shown for the illustrated embodiment. In FIG. 1, the endotracheal tube positioner 50 is illustrated as being removed from the endotracheal tube 20. In FIG. 2, the endotracheal tube positioner 50 is illustrated as being inserted into the endotracheal tube 20.

The endotracheal tube 20 comprises a proximal end 22 and a distal end 24. The distal end 24 is the end of the endotracheal tube 20 oriented most deeply inside the body of a patient when the endotracheal tube 20 is placed as designed into a patient's body during a procedure. The proximal end 22 is the end of the endotracheal tube 20 that is located farthest outside of a patient's body (or nearest the practitioner performing the procedure) when the endotracheal tube 20 is placed as designed into a patient's body during a procedure.

The endotracheal tube 20 illustrated in FIG. 1 includes a main body 26, an inflatable cuff 28, a main passageway 30, a cuff tube 31, a suction tube 34, and a suction opening 39.

The main body 26 is a generally tube shaped structure defining a main passageway 30 that extends therethrough. The main body 26 is sized and shaped so that it may be inserted into the trachea of a patient. An endotracheal tube used for a given patient is selected so that its length and diameter provide an appropriate match with the size and/or other particulars of the patient. When the endotracheal tube 20 is properly positioned in a patient, the distal tip of the distal end 24 is preferably located about 4 to about 6 centimeters above the carina (the branch point of the trachea), and the proximal end 22 extends outside of the patient. Thus, the main passageway 30 provides a conduit through which air may pass between the patient's trachea and the outside atmosphere.

The inflatable cuff 28 is located proximal to the distal end 24 of the endotracheal tube 20. The inflatable cuff 28 may be filled with air to an expanded position so that the inflatable cuff 28 provides a barrier to the passage of air between the wall of the trachea and the external surface of the main body 26 of the endotracheal tube 20. The inflatable cuff 28 may be deflated to a smaller position to facilitate intubation and extubation. Studies have shown that, for known cuffs currently in use, positioning of the cuff in the suprasternal notch offers a high degree of certainty that the tube is not positioned too deeply inside the patient. In the illustrated embodiment, the inflatable cuff 28, as well as the main body 26, are made of a material that will allow light to pass through. Light may, for example, also reflect off of the inflatable cuff 28.

The cuff tube 31 provides an air conduit for the inflation and deflation of the inflatable cuff 28. The cuff tube 31 includes a cuff lumen 33 extending through the cuff tube 31, providing a passageway for air between the inflatable cuff 28 and an external source of air for inflation and deflation of the inflatable cuff 28. The cuff tube 31 may be formed separately from the main body 26, integral with the main body 26, or a combination. At its proximal end, the cuff tube 31 includes a cuff adapter 32 that is adapted to allow a connection to an external source of air, such as a pump, and to allow and maintain inflation of the inflatable cuff 28.

The suction tube 34 provides a conduit for the removal of secretions that may accumulate above the inflatable cuff 28. The suction tube 34 includes a suction lumen 38 extending through the suction tube 34, providing a passageway for secretions to be removed. The suction lumen 38 is an example of an auxiliary passageway, that is, a passageway in addition to the main passageway (or passageways). The suction tube 34 may be formed separately from the main body 26, integral with the main body 26, or a combination. The main body 26 includes a suction opening 39 extending through the wall of the main body 26 and connecting with the suction lumen 38. At its proximal end, the suction tube 34 includes a suction adapter 36 that is adapted to allow a connection to an external source of suction, thereby allowing secretions to be removed from the trachea by being suctioned from the suction opening 39 via the suction lumen 38. Generally, the distance through the suction lumen 38 from the suction adapter 36 to the inflatable cuff 28 is from about 6 inches (approximately 15 centimeters) to about 9 inches (approximately 22.5 centimeters). An example of a known endotracheal tube providing a suction lumen is the Mallinckrodt Hi-Lo Evac® endotracheal tube.

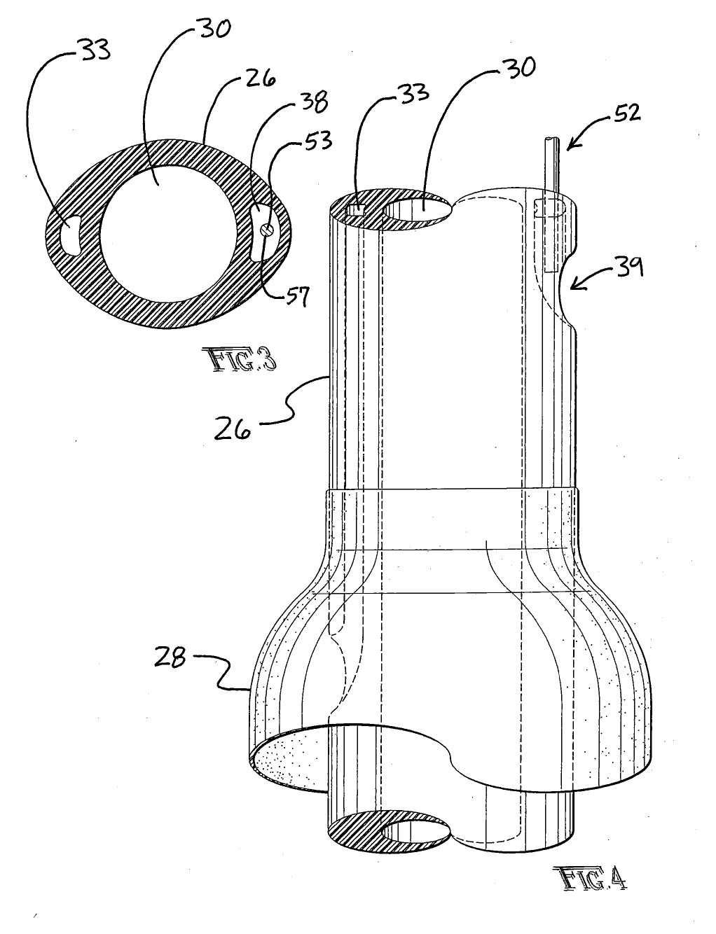

FIG. 3 illustrates a cross-section taken along line 3-3 of FIG. 1 (additionally illustrating when the endotracheal positioner 50 is in place). In the illustrated embodiment, at line 3-3, the suction lumen 38 and the cuff lumen 33 are located on opposite sides of the main passageway 30, extending through the wall of the main body 26 near the distal end 24 of the endotracheal tube 20. The suction lumen 38 may, for example, have a width of approximately 4 millimeters. The cuff lumen 33 may, for example, have a width of approximately 1.5 millimeters.

Returning to FIG. 1, as previously mentioned, the positioning system 40 includes a light source 42, a light funnel 44, and an endotracheal tube positioner 50. The light source 42 acts to provide light to the endotracheal tube positioner 50, which in turn will emit the light through the body of a patient to indicate the position of the endotracheal tube. The light source 42 may be, for example, a medical grade source, or, as an additional example, a penlight. The light from the light source should be of a type and intensity such that it may pass through the tissue of a patient (as well as portions of the endotracheal tube 20) when emitted from the endotracheal tube positioner 50, so that the light may be viewed from the exterior of a patient by a practioner. The light used may be, for example, white light, or, as an additional example, red light.

The light funnel 44 acts to assist in the transmission of light from the light source 42 to the endotracheal tube positioner 50. The light funnel 44, for example, may have a mirrored inner surface to assist in the efficient transmission of light to the endotracheal tube positioner 50.

The endotracheal tube positioner 50 includes a proximal end 58 and a distal end 60. The distal end 60 is the end is the end of the endotracheal tube positioner 50 oriented most deeply inside the body of a patient when the endotracheal tube positioner is placed as designed into the endotracheal tube 20 during a procedure. The proximal end 58 is the end of the endotracheal tube positioner 50 that is located farthest outside of a patient's body (or nearest the practitioner performing the procedure) when the endotracheal tube positioner 50 is placed as designed into the endotracheal tube positioner 50 during a procedure. The endotracheal tube positioner 50 includes a light source adapter 54, a body 55, a positioning body 52, and a positioning element 56.

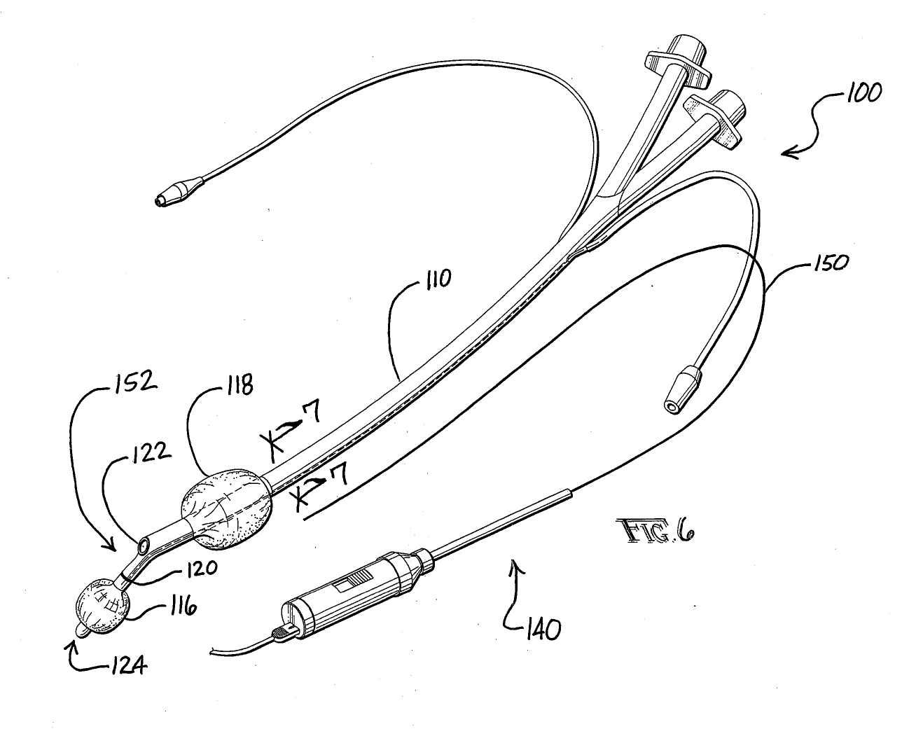

As best seen in FIG. 5, which illustrates a cross-section of the postioning system 40 taken along line 5-5 of FIG. 1, the light source adapter 54 (which is located at the proximal end of the endotracheal tube positioner 50) threadedly engages the light funnel 44, into which the light source 42 is placed (in the illustrated embodiment, the light source adapter 54 threadedly engages the light funnel 44). The positioning body 52 extends through the light source adapter 54, and is exposed to light from the light source 42 and light funnel 44. The engagement between the light source adapter 54 and light funnel 44 allows the light source 42 and light funnel 44 to be connected to and removed from the endotracheal positioner 50, thereby facilitating easier insertion (as well as removal) of the endotracheal positioner 50 into the endotracheal tube 20. The body 55 of the endotracheal tube positioner 50 extends from the light source adapter 54 and surrounds a portion of the positioning body 52. The body 55 is of a sufficient size and rigidity to allow for ease of handling the endotracheal tube positioner 50 during insertion into and removal from the endotracheal tube 20.

Returning to FIG. 1, as mentioned above, the endotracheal positioner 50 includes a positioning body 52 and a positioning element 56. The positioning element 56 is located proximal (i.e. at or near) to the distal end 60 of the endotracheal positioner 50. The positioning element, for example, may be integral to at least a portion of the positioning body 52. As an example of another alternative, the positioning element 56 may be formed separately from the positioning body 52. The positioning body 52 is of a size and flexibility so that the positioning body 52 may be inserted into and manipulated into and out of the suction lumen 38 while the endotracheal tube 20 is in place in a patient. In the illustrated embodiment, the positioning body 52 is a cable including a fiber optic channel 53 extending from the proximal end 58 to the distal end 60, thereby providing a conduit for light to travel from the light source 42 and light funnel 44 to the positioning element 56. Along a portion of the length of the positioning body 52, the fiber optic channel 53 is surrounded by an insulating sleeve 57 (see also FIG. 3). The insulating sleeve 57 acts to prevent the passage of light, thereby reducing losses as well as allowing illumination of a more specific portion of the endotracheal tube 20 when the endotracheal positioner 50 is in place. The insulating sleeve 57 may be, for example, from about 1 to about 5 millimeters thick. The positioning body 52 is sized and dimensioned so that it may be inserted into the suction lumen 38 and then advanced such that the proximal end 60 is located proximal (i.e. at or near) to the inflatable cuff 28 and/or the suction opening 39. For example, the endotracheal positioner 50 may have an overall length of about 40 to about 45 centimeters.

As indicated above, the insulating sleeve 57 does not cover the entire length of the positioning body 52. For example, in the illustrated embodiment, a length of the fiber optic channel 53 about 0.2 centimeters to about 0.5 centimeters closest to the distal end 60 of the positioning body 52 may be uninsulated and form the positioning element 56. As the positioning element 56 is not surrounded by insulation, light passes through the positioning element 56, which may be used by an observer to identify the position of the endotracheal tube 20 when the endotracheal positioner 50 is in place. Further, the positioning element 56 may include a roughened edge to allow a scattering of light to improve visibility of the emitted light.

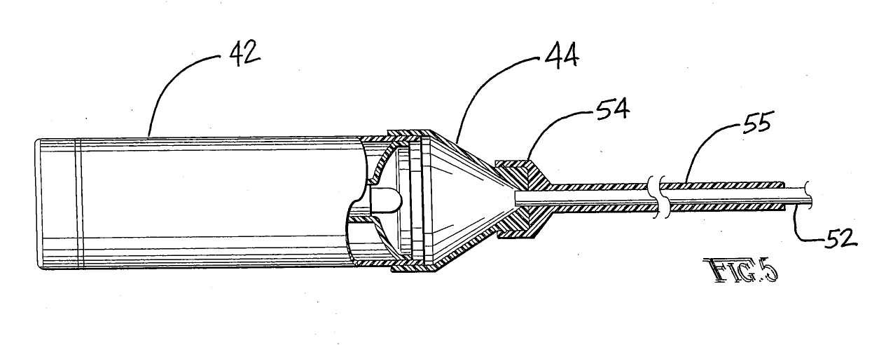

To use the endotracheal positioner 50, with the endotracheal tube 20 already placed in the patient (i.e. the patient is intubated), the distal end 60 of the endotracheal positioner 50 is inserted into the opening of the suction lumen 38 located proximal to the suction adapter 36. The positioning body 52 is then progressively advanced into the suction lumen 38 until the distal end 60 is proximal (i.e. at or near) the inflatable cuff 28, causing resistance to be felt. (See also FIGS. 2 and 4 illustrating the endotracheal tube system 10 with the positioning body 52 advanced into the suction lumen 38). Alternatively, positive mechanical stops and/or markings may be incorporated into other portions of the endotracheal tube system 10 to indicate how far the positioning body 52 should be advanced. For example, the endotracheal positioner 50 may be sized so that it is properly positioned when the body 55 contacts the suction adapter 36, preventing further insertion of the endotracheal positioner 50. Once the positioning body 52 is thus positioned, the light funnel 44 and light source 42 may be attached to the light source adapter 54. When the light source 42 is activated, light travels from the light source 42 through the light funnel 44 where it strikes the positioning body 52. The light is then sent to the positioning element 56 via the fiber optic channel 53. The light is then emitted through the positioning element 56 where it passes though the endotracheal tube 20, inflatable cuff 28 (emitted light may also reflect off of the inflatable cuff 28), and body tissue of the patient, and can be observed by a practitioner.

Because the endotracheal positioner 50 is placed such that the positioning element 56 is in a known position (e.g. the inflatable cuff 28 is illuminated), the position of the endotracheal tube 20 within the patient may be identified. For example, studies have shown that, for known cuffs currently in use, positioning of the cuff in the suprasternal notch offers a high degree of certainty that the tube is not positioned too deeply inside the patient. Thus, in the illustrated embodiment, if a practitioner can visually identify that the inflatable cuff 28 is located in the suprasternal notch, there is a high degree of certainty that the endotracheal tube 20 is properly positioned. The light source 42 and light funnel 44 may then be disconnected, and the endotracheal positioner 50 removed from the suction lumen 38. If the inflatable cuff 28 is not located in the suprasternal notch, then the endotracheal tube may be adjusted to the correct position. Should it be desirable to check and/or confirm that the endotracheal tube 20 has remained in the proper position (for example, if the patient's position has changed), the endotracheal positioner 50 may be re-inserted into the suction lumen 38 and the position of the endotracheal tube 20 checked again. As the endotracheal positioner 50 is removable, it is separately disposable from the endotracheal tube 20. Moreover, should any damage occur to the endotracheal tube 20 after it is positioned, the endotracheal positioner 50 will not be damaged and any resultant adverse consequences can be lessened.

The endotracheal positioner 50 of the illustrated embodiment may also be used during a tracheostomy procedure. With the light source 42 activated and light emitting from the positioning element 56, the endotracheal tube 20 may be retracted gradually, with the practitioner visually observing the position based on the light emitting through the patient. Because the positioning element 56 is located at a known position of the endotracheal tube 20, the practitioner can retract the endotracheal tube to the desired position. Then, based on the distance from the known position of the positioning element 56 to the tip of the distal end 24 of the endotracheal tube 20, the practioner can puncture and enter the trachea, without causing the patient safety issues as well as adverse time and cost consequences of piercing and damaging the endotracheal tube 20 and/or any other devices that may be used in a tracheostomy procedure.

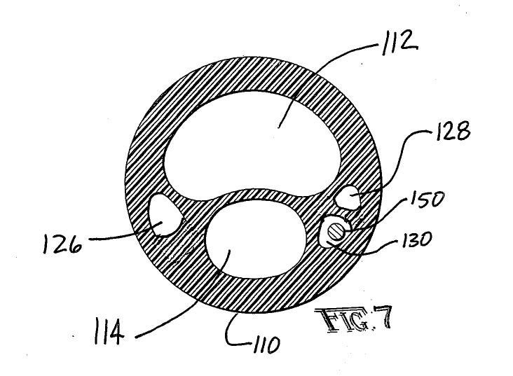

FIG. 6 illustrates additional aspects of at least one additional embodiment of the presently discussed technology. FIG. 6 (along with FIG. 7, which illustrates a cross-section taken along line 7-7) shows a double lumen endotracheal tube system 100 according to at least one embodiment of the present technology. The double lumen endotracheal tube system 100 includes a double lumen endotracheal tube 110 and a positioning system 140. Certain aspects of the double lumen endotracheal tube system 100 may be similar to the previously discussed exemplary embodiment.

The double lumen endotracheal tube 110 includes a bronchial lumen 112 and a tracheal lumen 114. As best seen in FIG. 6, the bronchial lumen 112 is longer than the tracheal lumen 114. The bronchial lumen 112 is sized and adapted to extend past the carina and into a main bronchus when the double lumen endotracheal tube 110 is in position, while the tracheal lumen 114 is sized and adapted to remain in the trachea. The bronchial lumen 112 and tracheal lumen 114 provide first and second air passageways. The double lumen endotracheal tube 110 also includes a bronchial cuff 116 and a tracheal cuff 118. The bronchial cuff 116 is located distal of the tracheal cuff 118 at the portion of the endotracheal tube where the bronchial lumen 112 extends past the tracheal lumen 114. The bronchial cuff 116 extends radially from the bronchial lumen 112 and is sized and adapted to be expandable to contact the walls of a main bronchus. The tracheal cuff 118 extends radially from both the bronchial lumen 112 and the tracheal lumen 114 and is sized and adapted to be expandable to contact the walls of the trachea. Located distal from the tracheal cuff 118 and proximal from the bronchial cuff 116 are a position indicator 120, and a tracheal opening 122. The tracheal opening 122 is located at the distal end of the tracheal lumen 114, and provides an opening to the trachea from the tracheal lumen 114. The position indicator 120 provides a cue for determining the position of the double lumen endotracheal tube 110. The position indicator 120 may be, for example, a line scribed on the exterior of the double lumen endotracheal tube 110 approximately 1 centimeter from the bronchial cuff 116. Further, the position indicator 120 may include a positive mechanical stop (associated with the auxiliary passageway 130, see below), which prevents pushing the distal end 152 of the endotracheal positioner 150 into the bronchial cuff 116. A bronchial opening 124 is located at the distal end of the bronchial lumen 112, and provides an opening to the bronchus from the bronchial lumen 112.

As best seen in FIG. 7, the cross-sectional areas of the bronchial lumen 112 and the tracheal lumen 114 may be generally similar. The double lumen endotracheal tube 110 includes a bronchial cuff lumen 126 and a tracheal cuff lumen 128, which provide an air conduit for the inflation and deflation of the bronchial cuff 116 and the tracheal cuff 118, respectively. The double lumen endotracheal tube 110 also includes an auxiliary passageway 130. The auxiliary passageway 130 extends distally to approximately the position indicator 120, where it may terminate at a positive mechanical stop (not shown). This positive mechanical stop can help prevent perforation of the endobronchial cuff 116 by the endotracheal positioner 150. The auxiliary passageway 130 may be, for example, between about 1 millimeter and about 1.5 millimeters in diameter.

The positioning system 140 includes a removable positioning member such as endotracheal positioner 150 having a distal end 152. The endotracheal positioner may be similar in many respects to the endotracheal positioner described previously. For example, the endotracheal positioner 150 may comprise a fiber optic channel that emits light from the distal end 152 of the endotracheal positioner 150. The endotracheal positioner 150 is sized and adapted to be insertable into and removable from the auxiliary passageway 130 (which may be, from above, between about 1 millimeter and about 1.5 millimeters in diameter). Hence, the endotracheal positioner 150 may have a smaller diameter than the endotracheal positioner previously described. As a result, the light emitted from the distal end 152 of the endotracheal positioner 150 may not be as strong, and may not be visible outside the body of the patient. The length of the endotracheal positioner 150 is selected so that the distal end 152 of the endotracheal positioner can be placed at or near to the position indicator 120 when the endotracheal positioner 150 is inserted into the auxiliary passageway 130.

To use the endotracheal positioner 150, with the double lumen endotracheal tube 110 already placed in the patient, the distal end of the endotracheal tube 150 is inserted into the opening of the auxiliary passageway 130, and extended until the distal end 152 of the endotracheal positioner 150 is at or near the position indicator 120. Once the endotracheal positioner 150 is in place as described, a light source may be provided. When the light source is activated, light travels to the distal end 152 of the endotracheal positioner 150, from where it is emitted. The emitted light then may emit through the tracheal opening 122, illuminating the position indicator 120 and/or illuminating the bronchial cuff 116. In the illustrated embodiment, the emitted light may not be visualized outside of the patient's body, and a fiberoptic bronchoscope is introduced into the double lumen endotracheal tube 110 to observe the position of the endotracheal tube 110. The depth of the double lumen endotracheal tube 110 may be adjusted accordingly, for example, until the light emitted can be seen just outside of the endobronchial opening into which the bronchial lumen 112 is inserted. The emitted light may also, for example, illuminate the bronchial cuff 116 to help determine proper positioning. Further, by illuminating the bronchial cuff 116, the endotracheal positioner 150 can also help a practitioner determine if there is a problem with the location of the bronchial cuff 166, such as potential cuff herniation.

As can be gathered from the foregoing, certain embodiments of the present technology thus can provide, for example, cost effective, dynamic, real time, visually verifiable non-radiologic endotracheal tube positioning. Further, certain embodiments of the present technology also reduce the number of moving parts; reduce the cost and complexity of endotracheal tube positioning; utilize disposable components and minimize cross contamination and infection risk; provide improved patient safety profile; reduce risks of heat or safety concerns of embedded electrical components; and/or provide for ease of use in endotracheal positioning. Also, certain embodiments of the present technology allow for checking the optimal position of an endotracheal tube; guiding endotracheal tube depth adjustment when a patient's position has been changed; ease of use in the field by emergency medical personnel; use for airway management when a radiology facility is not easily available; and/or dynamically guiding the positioning of an endotracheal tube while performing percutaneous tracheostomy procedures in the ICU.

While the present technology has been described with reference to certain embodiments, it will be understood by those skilled in the art that various changes may be made and equivalents may be substituted without departing from the scope of the invention. In addition, many modifications may be made to adapt a particular situation or material to the teachings of the technology without departing from its scope. Therefore, it is intended that the present technology not be limited to the particular embodiment disclosed, but that the presently described technology will include all embodiments falling within the scope of the appended claims.

1. An endotracheal tube system comprising:
an endotracheal tube for providing airway maintenance during an endotracheal procedure; and
a removable positioning member having a distal end, the removable positioning member having a positioning element located proximal to the distal end, and wherein the positioning element provides an indication of position.

2. The endotracheal tube system of claim 1, wherein the positioning element emits light visible through a patient's body.

3. The endotracheal tube system of claim 2, further comprising a light source attachable to and detachable from the removable positioning member.

4. The endotracheal tube system of claim 1 wherein the removable positioning member comprises a cable comprising a fiber optic channel.

5. The endotracheal tube system of claim 1 wherein the removable positioning member comprises a cable comprising a fiber optic channel and an insulation sleeve surrounding a portion of the fiber optic channel, the cable having an insulated portion through which light does not pass and an uninsulated portion through which light does pass, the uninsulated portion located proximal to the distal end of the positioning member.

6. The endotracheal tube system of claim 1, wherein the endotracheal tube comprises a main passageway and an auxiliary passageway, the main passageway sized and adapted for providing airway maintenance during an endotracheal procedure, the auxiliary passageway having a smaller cross sectional area than the main passageway, and wherein the removable positioning member comprises a cable sized and adapted to be insertable into and removable from the auxiliary passageway.

7. The endotracheal tube system of claim 6, wherein the endotracheal tube comprises an inflatable cuff, the inflatable cuff located proximal to a distal end of the endotracheal tube, and the removable positioning member is sized and adapted so that the distal end of the removable positioning member is positioned proximal to the inflatable cuff when the removable positioning member is fully inserted into the endotracheal tube.

8. The endotracheal tube system of claim 6, wherein the endotracheal tube comprises a cuff auxiliary passageway and an inflatable cuff, the inflatable cuff located proximal to a distal end of the endotracheal tube, the cuff auxiliary passageway providing an airway for inflating or deflating the inflatable cuff, the removable positioning member comprises a cable sized and adapted to be insertable into and removable from the auxiliary passageway, the positioning element adapted to emit light visible through a patient's body, and wherein the auxiliary passageway is located to provide an opening approaching the inflatable cuff such that light emitted from the positioning element passes through the inflatable cuff when the removable positioning member is fully inserted into the endotracheal tube.

9. The endotracheal tube system of claim 1, wherein the endotracheal tube comprises a first air passageway, a second air passageway, and an auxiliary passageway, the first and second air passageways sized and adapted for providing airway maintenance during an endotracheal procedure, the auxiliary passageway having a smaller cross sectional area than the main passageway, and wherein the removable positioning member comprises a cable sized and adapted to be insertable into and removable from the auxiliary passageway.

10. An endotracheal tube positioner comprising a positioning member sized and adapted to be insertable into and removable from an endotracheal tube, the positioning member having a distal end, the positioning member comprising a light emitting positioning element proximal to the distal end.

11. The endotracheal tube positioner of claim 10, wherein the positioning element emits light visible through the body of a patient when the positioning member is inserted into the endotracheal tube.

12. The endotracheal tube positioner of claim 10, wherein the positioning member is sized and adapted to be insertable into and removable from a suction lumen of the endotracheal tube.

13. The endotracheal tube positioner of claim 10, further comprising a light source attachable to and removable from the positioning member.

14. The endotracheal tube positioner of claim 10, wherein the positioning member is sized and adapted to extend proximal to an inflatable cuff when inserted into the endotracheal tube, and wherein the light emitted from the positioning element when the positioning member is inserted into the endotracheal tube passes through the inflatable cuff.

15. The endotracheal tube positioner of claim 10, wherein the positioning member comprises a cable comprising a fiber optic channel and an insulation sleeve surrounding a portion of the fiber optic channel, the cable having an insulated portion through which light does not pass and an uninsulated portion through which light does pass, the uninsulated portion located proximal to the distal end of the positioning member.

16. A method for identifying the position of an endotracheal tube in a patient's body, the method comprising the steps of:
advancing a removable positioning member through an auxiliary passageway into a known position in the endotracheal tube;
providing a light emitting from a distal portion of the removable positioning member;
observing the light emitted from the distal portion of the removable positioning member;
identifying the position of the endotracheal tube; and
removing the removable positioning member from the endotracheal tube.

17. The method of claim 16, wherein the step of observing the light emitted from the distal portion of the removable positioning member includes observing the light emitted through the body of the patient, and wherein the step of identifying the position of the endotracheal tube includes identifying the position of the endotracheal tube based on the location of the light emitted through the body of the patient.

18. The method of claim 16, wherein the known position is proximal to the location of an inflatable cuff located proximally to the distal end of the endotracheal tube.

19. The method of claim 17, wherein the endotracheal tube is positioned such that light emitting from the known position proximal to the location of the inflatable cuff passes through the body of the patient proximal to the patient's suprasternal notch.

20. The method of claim 16, further comprising:
adjusting the position of the endotracheal tube to accommodate a tracheostomy after identifying the position of the tracheal tube; and
puncturing the trachea at a distance away from the light emitted through the body of the patient.

21. The method of claim 16, wherein the removable positioning member is advanced into a suction lumen of the endotracheal tube.
